(19) United States
(12) Patent Application Publication
(10) Pub. No.: US 2012/0253461 A1
(43) Pub. Date: Oct. 4, 2012

(54) PROSTHETIC LIGAMENT FOR TRANSVERSE FIXATION AND METHOD FOR MAKING IT

(76) Inventors: Bernard Brulez, Bourbonne Les Bains (FR); Jacques-Philippe Laboureau, Le Tignet (FR)

(21) Appl. No.: 13/395,104

(22) PCT Filed: Jul. 31, 2009
(86) PCT No.: PCT/FR2010/000557

§ 371 Date: May 15, 2012

(30) Foreign Application Priority Data
Sep. 3, 1990 (FR) 0903790

(51) Int. Cl. A61F 2/08

(52) U.S. Cl. 623/13.12; 493/405

(57) ABSTRACT

A prosthetic ligament for replacing a natural articular ligament, comprising a first intraosseous end part, called the tibial end part, and a second intraosseous end part, called the femoral end part, the two intraosseous end parts surrounding an intraarticular central part, characterized in that the first intraosseous end part is in the form of two cylindrical strands, and in that the second intraosseous end part forms a loop connected to the first intraosseous end part via the intraarticular central part, which is composed of at least two bundles of technical filaments, each of the bundles being connected to the first intraosseous end part at one end of its ends and to the second intraosseous end part at the other of its ends.

### CROSS-REFERENCE TO RELATED APPLICATION

This application is a national stage application and claims the benefit of the priority filing date in PCT/FR2010/000557 referenced in WIPO Publication WO/2011/012783. The earliest priority date claimed is Jul. 31, 2009.

### FEDERALLY SPONSORED RESEARCH

None

### SEQUENCE LISTING OR PROGRAM

None

### STATEMENT REGARDING COPYRIGHTED MATERIAL

Portions of the disclosure of this patent document contain material that is subject to copyright protection. The copyright owner has no objection to the facsimile reproduction by anyone of the patent document or the patent disclosure as it appears in the Patent and Trademark Office file or records, but otherwise reserves all copyright rights whatsoever.

### BACKGROUND

The invention concerns a prosthetic ligament designed to be put in place by a transverse fixation technique as well as a method for making such a ligament. This ligament can be used in plastic surgery of the knee for the replacement of the anterior or posterior cruciform ligaments.

Known are a certain number of artificial ligaments designed to replace joint ligaments. Among these are ligaments obtained by coiled rolling or folding of textile ribbons of a biocompatible woven or knitted material in order to leave free, or on the other hand to interconnect, the longitudinal technical threads of the ligament. These known ligaments generally have an overall shape of an elongated cylinder comprising between two external intra-osseous parts and a middle intra-articular part. Examples of such ligaments are also provided in French patents FR 2155.846 and FR 2.688.690.

The customary surgical technique for fixation of these artificial ligaments or for biological ligament reconstruction involves boring a bone tunnel in the femur and in the tibia, then locking the ligament construction in these tunnels at its ends by an interference screw fixed frontally to the bone. To enable placement in the bone tunnels, the ends of the ligaments are provided with traction threads. Furthermore, at times, one has observed a sliding of the fixation screw on the prosthetic ligament in the area of the femur bone with this technique.

Surgeons have thus developed a new technique for implanting ligaments with a transverse fixation system, in which the femoral tunnel is a blind hole into which there emerges a lateral tunnel, enabling the insertion of a locking screw for the femoral end of the ligament. This allows a fixation with suspension and possibly with compression of the ligament. This new technique for fixation of the ligaments for ligament plastic surgery affords—besides a better traction strength of the ligament—the benefit of less trauma to the patient. This is so because there is no longer any need to bore a tunnel emerging into the bone of the femur. Furthermore, it improves the fibroblast colonization of the biological prostheses fixed in this manner.

Patents EP 1 493 404 and EP 0 145 492 describe artificial ligaments provided with eyelets at the end of their external intra-osseous parts. These eyelets serve to receive a fixation device, of a screw or knob type, at the external surface of the bone (the rest of the ligament passing through transverse bone tunnels). Thus, these ligaments are neither designed nor adapted to the transverse fixation technique.

Therefore, the purpose of the present invention is to provide an artificial prosthetic ligament adapted to these new techniques of transverse fixation, as well as a method of making such a ligament.

As such, the invention concerns a prosthetic ligament for the replacement of a natural joint ligament comprising a first intra-osseous or tibial end part, and a second intra-osseous or femoral end part, the two intra-osseous end parts flanking an intra-articular middle part. Said ligament being obtained by coiled rolling or folding of textile ribbons of a biocompatible material having longitudinal technical threads, wherein the first intra-osseous end part is in the form of two cylindrical strands and the second intra-osseous end part forms a loop connected to the first intra-osseous end part by the intra-articular middle part, which is made from at least two bundles of technical threads. Each of the bundles being joined at one of its ends to the first intra-osseous end part and by the other of its ends to the second intra-osseous end part.

One will easily grasp the advantage of this loop construction with a double bundle intra-articular middle part. This makes it possible to use a width having overall fewer technical threads as compared to the prosthetic ligaments of the prior art in the area of the first intra-osseous end part, which corresponds to the part fixed in the tibia, while the separation of the threads in the intra-articular middle part further improves the traction strength. The loop thus formed can be easily inserted and contained in the femoral half-tunnel made in this lateral fixation technique.

Preferably and advantageously, the technical threads are joined together in at least one of the first and second intra-osseous end parts and they are free in the intra-articular part.

According to the embodiment of the invention, the loop of the ligament is open.

The invention also concerns a method of making a ligament adapted to the transverse fixation technique with lateral tunnel.

This method is distinguished in that it involves at least the stages or providing a width having five consecutive sectors: a first woven end sector whose length corresponds to that of the tibial tunnel, a first intra-articular middle sector, a second woven end sector that will serve to form the second intra-osseous end part of the finished ligament, a second intra-articular middle sector, and a last woven end sector whose length corresponds to that of the tibial tunnel; then rolling the width onto itself to form a cylinder, having conical ends after slicing the ends on a slant; then fixing the coiled roll by at least one longitudinal stitching, performed at least on the first and last woven end sectors; and finally folding the cylindrical roll thus obtained into two, along its length, symmetrically, so as to form the open loop.

Other advantages and characteristics will better emerge from the following description of a sample embodiment (though not limited to this) of a ligament according to the invention and the method of making such a ligament.

### SUMMARY

The invention concerns a prosthetic ligament for the replacement of a natural joint ligament comprising a first intra-osseous or tibial end part, and a second intra-osseous or femoral end part, the two intra-osseous end parts flanking an infra-articular middle part. Said ligament being obtained by coiled rolling or folding of textile ribbons of a biocompatible material having longitudinal technical threads, wherein the first intra-osseous end part is in the form of two cylindrical strands and the second intra-osseous end part forms a loop connected to the first intra-osseous end part by the intra-articular middle part, which is made from at least two bundles of technical threads. Each of the bundles being joined at one of its ends to the first intra-osseous end part and by the other of its ends to the second intra-osseous end part.

This method is distinguished in that it involves at least the stages or providing a width having five consecutive sectors: a first woven end sector whose length corresponds to that of the tibial tunnel, a first intra-articular middle sector, a second woven end sector that will serve to form the second intra-osseous end part of the finished ligament, a second intra-articular middle sector, and a last woven end sector whose length corresponds to that of the tibial tunnel; then rolling the width onto itself to form a cylinder, having conical ends after slicing the ends on a slant; then fixing the coiled roll by at least one longitudinal stitching, performed at least on the first and last woven end sectors; and finally folding the cylindrical roll thus obtained into two, along its length, symmetrically, so as to form the open loop.

### FIGURES

FIG. 1 is a schematic and perspective top view representation of the ligament according to the invention,

FIGS. 2A to 2D represent the schematic stages of the method of making the ligament shown in FIG. 1.

### DETAILED DESCRIPTION

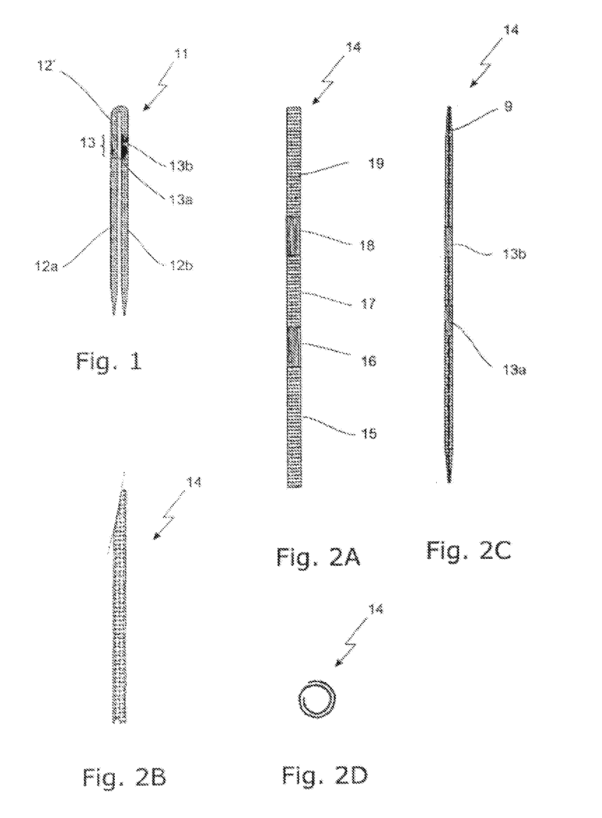

Referring to FIGS. 1 and 2A to 2D, we shall describe one mode of implementation of the ligament 11 according to the invention, known as an open loop.

This embodiment has numerous advantages: first, it is easy to fabricate. Moreover, as the final diameter of the bundles are narrow, it requires boring a small diameter tunnel, which is clearly preferable in terms of surgery.

According to one essential characteristic, referring to FIG. 1, the loop formed by the second intra-osseous end part 12′, or femoral end, is open. Thus, the first intra-osseous end part 12, or tibial end, is constituted of two cylindrical strands 12a, 12b.

The two intra-osseous end parts 12, 12′ are connected to each other via an intra-articular middle part 13 formed from two bundles 13a, 13b of technical threads. The technical threads of the intra-articular middle part 13 are free, whereas they are joined together in the intra-osseous end parts 12, 12′. It goes without saying that one could produce ligaments 11 according to the invention having technical threads joined together in the intra-articular middle part 13, without leaving the scope of the present invention.

This ligament 11 thus has five consecutive pieces: a first woven piece, corresponding to the cylindrical strand 12a, a second piece of free threads, corresponding to the bundle 13a, a third woven piece, corresponding to the first intra-osseous end part 12, a fourth piece of free threads, corresponding to the second bundle 13b and a fifth woven piece, corresponding to the cylindrical strand 12b.

Preferably, the ligament 11 has an axis of symmetry, such that the two cylindrical strands 12a, 12b are of the same length and respectively, the two bundles 13a, 13b.

This ligament 11 is specifically designed to be put into place by pushing in the bone tunnels by means of a placement guide that is the subject of a patent application not yet published, and filed by the applicant. Thus, it does not have any traction threads.

We shall now describe an example of the method of making this open-loop ligament 11 according to the invention, referring to the sequence illustrated in FIGS. 2A to 2D.

The ligament 11 is made from a single width 14, shown in FIG. 2A.

This width 14 has five consecutive sectors: a first woven end sector 15 whose length corresponds to that of the tibial tunnel, a first intra-articular middle sector 16, a second woven end sector 17 that will serve to form the second intra-osseous end part 12′ of the finished ligament 11, a second intra-articular middle sector 18, and a last woven end sector 19 whose length corresponds to that of the tibial tunnel.

As an example, but not limited to this, the length of the second woven end sector 17 should be around 35 mm, that of the first and second intra-articular middle sectors 16. 18 should be around 28 mm, and that of the first and last woven end sectors 15, 19 is respectively around 175 mm,

According to the preferred embodiment shown in FIGS. 1 and 2A to 20, the first and second intra-articular middle sectors 16, 18 are each composed solely of one skein of free longitudinal technical threads, whereas in the woven end sectors 15, 17, 19 the technical threads are joined together in a woven or knitted weft.

The first stage of the process, not shown in the figures, thus consists in cutting and/or knitting the width 14 to the required dimensions

One then rolls the width 14 onto itself, in a rotation of one turn and three-quarters of a turn, approximately, for example, in counterclockwise direction, as shown in FIG. 2D. During the rolling into a cylinder, one should take care to roll the free ends of the first and last woven end sectors 15, 19 into a conical shape, after having sliced the ends on a slant, as illustrated in FIG. 2B; then, referring to FIG. 2C, one fixes the coiled roll by a longitudinal stitching 9, made on all the woven end sectors 15, 17, 19 and on at least the first and the last end sectors 15, 19.

Finally, one folds the cylindrical roll thus obtained into two, along its length, symmetrically, so as to form the open loop.

It goes without saying that this method is given as an example and is only a preferred method of obtaining the ligament for transverse fixation according to the invention. The skilled person could make improvements here, especially in the order of the stages or the number of stitches, without thereby leaving the scope of the present invention.

Furthermore, while the specific technique for the placement of this new prosthetic ligament makes possible a significant improvement in its tissue colonization, one could of course apply specific treatments to the materials making up said ligament in order to improve its biocompatibility. In particular and advantageously, one could use the method of surface treatment as described in European patent EP 1.599.238 in the name of the applicant.

Finally, it goes without saying that, while the new artificial ligament and its method of fabrication are particularly intended for the replacement of the anterior cruciform ligament of the knee, this ligament structure can be used for the replacement of other ligaments and the examples just given are thus only particular illustrations, and in no case limiting of the areas of application of the invention.

1. A Prosthetic ligament (11) for the replacement of a natural joint ligament comprising a first intra-osseous or tibial end part (12), and a second intra-osseous or femoral end part (12′), the two intra-osseous end parts (12, 12′) flanking an intra-articular middle part (13), said ligament (11) being obtained by coiled rolling or folding of textile ribbons of a biocompatible material having longitudinal technical threads, wherein the first intra-osseous end part (12) is in the form of two cylindrical strands (12a, 12b) and the second intra-osseous end part (12′) forms an open loop connected to the first intra-osseous end part (12) by the intra-articular middle part (13), which is made from at least two bundles (13a, 13b) of technical threads, each of the bundles (13a, 13b) being joined at one of its ends to the first intra-osseous end part (12) and by the other of its ends to the second intra-osseous end part (12′), the ligament (11) thus consisting of five consecutive pieces: a first woven piece, corresponding to the cylindrical strand (12a), a second piece of free threads, corresponding to the bundle (13a), a third woven piece, corresponding to the first intra-osseous end part (12), a fourth piece of free threads, corresponding to the second bundle (13b) and a fifth woven piece, corresponding to the cylindrical strand (12b).

2. The Ligament (11) according to claim 1, wherein the second intra-osseous end part (12′) has a number of longitudinal technical threads equal to the sum of that of the bundles (13a, 13b) of threads of the intra-articular middle part (13).

3. The Ligament (1, 11) according to claim 1, wherein the technical threads are joined together in at least one of the first and second intra-osseous end parts (12, 12′) and they are free in the intra-articular middle part (13),

4. The Ligament (1, 11) according to claim 1, wherein the ligament (11) has an axis of symmetry, such that the two cylindrical strands (12a, 12b) are of the same length and respectively, the two bundles (13a, 13b).

5. A Method of making a ligament (11) according to claim 1, wherein it involves at least the stages of providing a width (14) having five consecutive sectors: a first woven end sector (15) whose length corresponds to that of the tibial tunnel, a first intra-articular middle sector (16), a second woven end sector (17) that will serve to form the second intra-osseous end part (12′) of the finished ligament (11), a second intra-articular middle sector (18), and a last woven end sector (19) whose length corresponds to that of the tibial tunnel; then rolling the width (14) onto itself to form a cylinder, having conical ends after slicing the ends on a slant; then fixing the coiled roll by at least one longitudinal stitching (9), performed at least on the first and last woven end sectors (15, 17); and finally folding the cylindrical roll thus obtained into two along its length, symmetrically, so as to form the open loop.
